SEQUENCE LISTING (13 sequences)

SEQ ID NO: 1 - DNA, 20 bp, Artificial Sequence
  Foxn1 forward oligonucleotide
cctgggttcagaggtcaaag

SEQ ID NO: 2 - DNA, 20 bp, Artificial Sequence
  Foxn1 reverse oligonucleotide
gggaaggctcccagttttac

SEQ ID NO: 3 - DNA, 20 bp, Artificial Sequence
  AFF1 forward oligonucleotide
tccacagtcccttccagaac

SEQ ID NO: 4 - DNA, 20 bp, Artificial Sequence
  AFF1 reverse oligonucleotide
cgctgtcacttgaactgctc

SEQ ID NO: 5 - DNA, 20 bp, Artificial Sequence
  AFF2 forward oligonucleotide
gcccctaggaaagaaccaag

SEQ ID NO: 6 - DNA, 20 bp, Artificial Sequence
  AFF2 reverse oligonucleotide
tgttgctgccactgctactc

SEQ ID NO: 7 - DNA, 20 bp, Artificial Sequence
  AFF4 forward oligonucleotide
agacagtgatggggaacagg

SEQ ID NO: 8 - DNA, 20 bp, Artificial Sequence
  AFF4 reverse oligonucleotide
tgcctcactgtcactggaac

SEQ ID NO: 9 - DNA, 20 bp, Artificial Sequence
  bFGF forward oligonucleotide
cctcacatcaagctacaact

SEQ ID NO: 10 - DNA, 20 bp, Artificial Sequence
  bFGF reverse oligonucleotide
acactcatccgtaacacatt

SEQ ID NO: 11 - DNA, 20 bp, Artificial Sequence
  GAPDH forward oligonucleotide
gagtcaacggatttggtcgt

SEQ ID NO: 12 - DNA, 20 bp, Artificial Sequence
  GAPDH reverse oligonucleotide
tgggatttccattgatgaca

SEQ ID NO: 13 - DNA, 22 bp, Artificial Sequence
  T7 polymerase sequence
taatacgactcactatagggag

(19) United States
(12) Patent Application Publication
(10) Pub. No.: US 2011/0076303 A1
(43) Pub. Date: Mar. 31, 2011

(54) Methods for Screening for Anti-Graying Agents on the Basis of AFF-4

(76) Inventors: Tokuro Iwabuchi, Kanagawa (JP); Lorin Weiner, Brooklyn, NY (US); Janice Brissette, Brooklyn, NY (US); Jian Li, Beijing (CN)

(21) Appl. No.: 12/823,606

(22) Filed: Jun. 25, 2010

Related U.S. Application Data
(60) Provisional application No. 61/220,538, filed on Jun. 25, 2009.

(51) Int. Cl. A61K 8/99; A61K 8/97; A61Q 5/00; A61P 17/00; A61K 36/06; A61K 36/258; A61K 36/899; A61K 36/00; C12Q 1/02; C12Q 1/68

(52) U.S. Cl. 424/195.15; 424/728; 424/750; 424/725; 435/29; 435/6

(57) ABSTRACT

An objective of the present invention is to provide an anti-graying agent. The present invention provides a method for screening an anti-graying agent whereby a substance that increases expression of AFF-4 in cells is selected by applying a candidate substance to cells in vitro; and, an anti-graying agent containing at least one type of herbal extract selected from the group consisting of Ganoderma lucidum (Fr.) Karst. extract, Panax schinseng Nees extract, Oryza sativa (Rice) Bran extract and Rabdosia japonicus extract selected with this method in an amount effective for increasing expression of AFF-4.

### CLAIM OF PRIORITY

This application claims priority under 35 USC §119(e) to U.S. Patent Application Ser. No. 61/220,538, filed on Jun. 25, 2009, the entire contents of which are hereby incorporated by reference.

### TECHNICAL FIELD

The present invention provides a method for screening anti-graying agents using AFF-4 as an indicator, and an anti-graying agent screened by that method.

### BACKGROUND

Melanin pigment responsible for determining hair color is supplied to hair after being biosynthesized from tyrosine in melanosomes within melanocytes (melanin-synthesizing cells) present in the upper portions of the hair bulb. Although gray hair is thought to involve a reduction in melanocytes or melanosomes, a decrease in the amount or activity of tyrosinase caused by abnormalities in these cells or organs or impairment of melanosome transport attributable to factors such as aging and stress, the entire mechanism behind this is not clear.

Numerous substances have previously been reported to have anti-graying action such as prevention of graying or inhibition of graying, and although, for example, this type of anti-graying action has been reported in Japanese pepper (Zanthoxylum piperitum) extract (see Japanese Unexamined Patent Publication No. H11-5720), there is a desire to enrich active ingredients having equal or better anti-graying action.

Pigment formation in pigment cells is known to be increased following activation of a transcription factor in the form of Foxn1 (see Weiner, et al., (2007) Cell, 130: 932-942, Dedicated Epithelial Recipient Cells Determine Pigmentation Patterns). Foxn1 is a transcription factor present in epithelial cells that controls expression of bFGF

### SUMMARY

An objective of the present invention is to elucidate the mechanism of pigment formation in follicular pigment cells, and to provide one or more effective anti-graying agent not found in the prior art as well as anti-graying compositions containing the same.

The inventors of the present invention found that molecular AFF-4 present in follicular epithelial cells binds to Foxn1 and promotes the transcription activity thereof, and that AFF-4 is not present in the epidermis in the case of humans, but rather is only expressed in epithelial cells of hair follicles. Moreover, AFF-4, Foxn1 and bFGF were all found to be clearly decreased in human gray hair. On the basis of these results, it was shown for the first time that substances are able to act only on pigment cells present in hair follicles mediated by AFF-4.

In order to find an embodiment thereof, a search was made for substances able to induce expression of AFF-4 using epithelial cells of human hair follicles. As a result, Ganoderma lucidum (Fr.) Karst. extract, Panax schinseng Nees extract, Oryza sativa (Rice) Bran extract, Rabdosia japonicus extract and Zanthoxylum piperitum extract were found to increase expression of AFF-4. Moreover, bFGF expression-increasing effects in human follicular epithelial cells have been confirmed for Ganoderma lucidum (Fr.) Karst. extract and Zanthoxylum piperitum extract. Accordingly, a hypothesis advocating signal transfer from AFF-4 to Foxn1 and then to bFGF in human follicular epithelial cells has been verified. When the bFGF expression-increasing effects of Ganoderma lucidum (Fr.) Karst. extract and Zanthoxylum piperitum extract were attempted to be verified in human epidermal cells, bFGF expression-increasing effects were not observed in the same cells for any of the substances tested, thereby suggesting that AFF-4 expression increase and bFGF expression increase in human follicular epithelial cells are phenomena that are specific to these cells.

On the basis of the above, it became possible to use the aforementioned substances to specifically activate follicular pigment cells by means of follicular epithelial cells adjacent thereto.

Thus, in a first aspect thereof, the present invention provides methods of screening for anti-graying agents. The screening methods are used to select and/or identify substances that increase expression of AFF-4 in cells by applying a candidate substance to the cells and optionally evaluating the expression of AFF-4 in the cells. In some embodiments, the cells are follicular epithelial cells.

In some embodiments thereof, the increase of expression of AFF-4 in the cells is evaluated by measuring an amount of mRNA encoding AFF-4 extracted from the cells by, for example, PCR and real-time PCR.

In a second aspect thereof, the present invention provides a substance screened with the aforementioned screening method, and more specifically, provides an anti-graying agent containing at least one type of herbal extract selected from the group consisting of Ganoderma lucidum (Fr.) Karst. extract, Panax schinseng Nees extract, Oryza sativa (Rice) Bran extract and Rabdosia japonicus extract in an amount effective for increasing expression of AFF-4 in cells, e.g., in follicular epithelial cells.

In a third aspect thereof, the present invention provides a method for increasing expression of AFF-4 in cells, e.g., follicular epithelial cells, using a substance selected with the aforementioned screening method, and more specifically, using at least one type of herbal extract selected from the group consisting of Ganoderma lucidum (Fr.) Karst. extract, Panax schinseng Nees extract, Oryza sativa (Rice) Bran extract, Rabdosia japonicus extract and Zanthoxylum piperitum extract.

In a fourth aspect thereof, the present invention provides a method for preventing or inhibiting gray hair comprising: increasing expression of AFF-4 by coating onto the scalp a substance selected with the aforementioned screening method, and more specifically, at least one type of herbal extract selected from the group consisting of Ganoderma lucidum (Fr.) Karst. extract, Panax schinseng Nees extract, Oryza sativa (Rice) Bran extract, Rabdosia japonicus extract and Zanthoxylum piperitum extract, in an amount effective for increasing expression of AFF-4 in cells, and preferably follicular epithelial cells.

In a fifth aspect thereof, the present invention provides the use of at least one type of herbal extract selected from the group consisting of Ganoderma lucidum (Fr.) Karst. extract, Panax schinseng Nees extract, Oryza sativa (Rice) Bran extract, Rabdosia japonicus extract and Zanthoxylum piperitum extract, in an amount effective for increasing expression of AFF-4 in cells in the preparation of a pharmaceutical or cosmetic composition for preventing or inhibiting gray hair.

In a sixth aspect thereof, the present invention provides a composition for increasing expression of AFF-4 in cells, e.g., follicular epithelial cells, comprising a substance selected with the aforementioned screening method, and more specifically, comprising at least one type of herbal extract selected from the group consisting of Ganoderma lucidum (Fr.) Karst. extract, Panax schinseng Nees extract, Oryza sativa (Rice) Bran extract, Rabdosia japonicus extract and Zanthoxylum piperitum extract. The composition can be a pharmaceutical or a cosmetic composition.

In a seventh aspect thereof, the present invention provides a composition for preventing or inhibiting gray hair that includes a substance selected with the aforementioned screening method, and more specifically, at least one type of herbal extract selected from the group consisting of Ganoderma lucidum (Fr.) Karst. extract, Panax schinseng Nees extract, Oryza sativa (Rice) Bran extract, Rabdosia japonicus extract and Zanthoxylum piperitum extract, in an amount effective for accelerating expression of AFF-4 in cells, e.g., follicular epithelial cells. The composition can be a pharmaceutical or a cosmetic composition.

According to the present invention, novel anti-graying agents can be identified by screening, and anti-graying agents can be provided that can be considerably more advantageous than those of the prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 shows the Foxn1 transcription activity-promoting effect of AFF-4.

FIG. 2 shows binding of AFF-4 to Foxn1.

FIG. 3 shows expression of Foxn1, AFF-1, AFF-2 and AFF0-4 in human anagen hair follicles.

FIG. 4 shows expression of Foxn1 and AFF-4 in human anagen hair follicles as determined by immunostaining and in situ hybridization.

FIG. 5 shows a comparison of the degrees of expression of Foxn1, AFF-1, AFF-2 and AFF-4 in human anagen hair follicles.

FIG. 6 shows a comparison of the expression levels of Foxn1, AFF-4 and bFGF between human black hair and human gray hair.

FIG. 7 shows the effect of Ganoderma lucidum (Fr.) Karst. extract on AFF-4 expression by human ORS as investigated over time.

FIG. 8 shows the effect of Zanthoxylum piperitum extract on AFF-4 expression by human ORS as investigated over time.

FIG. 9 shows the effect of Panax schinseng Nees extract on AFF-4 expression by human ORS.

FIG. 10 shows the effect of Oryza sativa (Rice) Bran extract on AFF-4 expression by human ORS.

FIG. 11 shows the effect of Rabdosia japonicus extract on AFF-4 expression by human ORS.

FIG. 12 shows the effect of Ganoderma lucidum (Fr.) Karst. extract on bFGF expression by human ORS.

FIG. 13 shows the effect of Zanthoxylum piperitum extract on bFGF expression by human ORS.

FIG. 14 shows the effect of Ganoderma lucidum (Fr.) Karst. extract and Zanthoxylum piperitum extract on bFGF expression in human epidermal cells (HaCaT).

### DETAILED DESCRIPTION

As noted above, the present invention is based, in part, on applicants' studies of gene expression in follicular pigment cells. Accordingly, the invention features compositions, including the extracts described herein, which can be used to maintain or produce pigment in hair. Other compositions within the invention include nucleic acid primers and probes that can be used to detect expression of an AFF-4 gene, reporter constructs (e.g., an AFF-4 regulatory sequence operably linked to a sequence encoding a readily detectable protein such as a fluorescent protein or enzyme), cells, cellular arrays, or tissue explants including epithelial follicular cells or cells engineered to include an AFF-4 reporter construct, and kits including one or more of these compositions.

The anti-graying agents described herein or discovered by the present screening methods may be agents that increase expression of AFF-4 by acting on the endogenous AFF-4 gene to increase the rate at which transcription occurs and/or to increase the resulting amount of mRNA. The invention is not, however, limited to agents that affect AFF-4 expression by any particular mechanism. For example, the screening methods can also be configured to identify anti-graying agents that increase the amount of AFF-4 protein and/or the activity of that protein. Any method known in the art for analyzing RNA or protein expression can be used in the context of the present methods, whether the expression is of an endogenous gene or a reporter gene construct.

Analysis of AFF-4 expression can be carried out in vitro or in cell-based screening assays. For example, follicular epithelial cells can be arrayed, and populations within the array can be contacted with one or more candidate anti-graying agents. More specifically, the present methods for identifying an anti-graying agent can include the steps of contacting a cell (e.g., a follicular epithelial cell) with a test substance or candidate agent; measuring or otherwise analyzing expression of AFF-4 in the cell; and determining whether the test substance or candidate agent increases the expression of AFF-4 relative to a control (e.g., relative to the level of expression in a cell that has not been exposed to the test substance). The cell can be maintained in tissue culture or can be a cell in vivo. Alternatively, or in addition, one can assess the ability of the test substance or candidate agent to maintain or increase the pigmentation in hair.

Anti-graying agents can be nucleic acids that encode AFF-4.

AFF-4 and Foxn1

AFFs are nuclear proteins belonging to the AF4/FMR2 family, and although they are thought to be activating factors of intranuclear transcription, the details thereof are not fully understood (Berton, et al. (1996) Nutr. Cancer 26: 353-363). Four types of AFF are known in humans, referred to as hAFF-1 (GenBank NM_{—}005935.2), hAFF-2 (GenBank NM_{—}002025.2), hAFF-3 (GenBank NM_{—}002285.2) and hAFF-4 (GenBank NM_{—}014423.3), and their genes have also been reported. AFF-3 is believed to be expressed in the central nervous system.

Foxn1 is also thought to be a nuclear protein that serves as a kind of transcription factor. It was previously referred to as Whn, and was discovered as a causative gene of nude mice. Foxn1 is known to play an important role in the formation of hair shafts, and has been said to be involved in hair formation (Brissette, et al. (1996) Genes Dev. 10: 2212-2221). In this patent, Foxn1 was clearly determined to be involved in not only hair formation, but also in hair pigment formation mediated by AFF4.

Anti-Graying Agent Screening Method

The present invention provides a method for screening anti-graying agents. This method is comprised of evaluating the ability of a candidate substance to increase expression and/or activity of AFF-4, and then select the candidate substance having such ability as an anti-graying agent.

More specifically, the screening methods include applying candidate substances to cells, evaluating expression of AFF-4 in the cells, and selecting those substances that increase expression of AFF-4 in the cells. In some embodiments, the cells are follicular epithelial cells. The cells may be of human origin or non-human origin, examples of which include cells of various mammals such as rats, mice or rabbits.

Candidates to be tested in the screening methods described herein include crude or purified extracts of organic sources, e.g., animal or herbal extracts, as well as partially or fully purified or synthetic agents, e.g., small molecules, polypeptides, lipids and/or nucleic acids, and libraries of any of the above.

Expression of the aforementioned physiologically active substance in the skin can also be determined by, for example, extracting total RNA from skin cells and measuring the amount of mRNA encoding that physiologically active substance. Extraction of mRNA and measurement of the amount thereof are commonly known in the art, and for example, quantification of RNA is carried out by a quantitative polymerase chain reaction (PCR) method. In addition, since the nucleotide sequences of genes encoding human AFF-4 are known, primers suitable for amplification of each gene can be suitably selected by a person with ordinary skill in the art based on that nucleotide information. Although suitable primers for real-time PCR amplification of AFF-4 are respectively used in the examples of the present description, the amplification primers are not limited thereto.

In addition, expression of the aforementioned physiologically active substances can also be determined by directly measuring the amount of the physiologically active substances in cells. This measurement can be carried out using various methods, examples of which include methods commonly known in the art utilizing antibody specific to the physiologically active substance, immunostaining methods utilizing a fluorescent substance, pigment or enzyme, Western blotting and immunoassays such as ELISA or RIA.

The present invention provides an anti-graying agent containing a substance selected with the aforementioned screening method, and more particularly, at least one type of herbal extract selected from the group consisting of Ganoderma lucidum (Fr.) Karst. extract, Panax schinseng Nees extract, Oryza sativa (Rice) Bran extract and Rabdosia japonicus extract.

A herbal extract used in the present invention refers to various types of solvent extracts, diluted solutions thereof, concentrates thereof or dried powders thereof obtained by crushing the herb followed by either extracting with a solvent at normal temperature or while heating or extracting using an extractor such as a Soxhlet extractor.

Although an herbal extract obtained according to the aforementioned extraction methods can be used directly in the form of an extraction solution as an active ingredient of the anti-graying agent of the present invention, as was previously described, the extract can also be used after diluting, concentrating or preparing in the form of a powder or paste after freeze-drying. In addition, inactive contaminants can also be removed from the extract using a technique such as liquid-liquid partitioning, and use of such an extract is contemplated in the present invention.

Since each of the aforementioned herbal extracts is as previously described, they can be acquired in accordance with ordinary methods, and can be obtained using the aforementioned techniques or instruments by, for example, immersing or heat-refluxing with an extraction solvent followed by filtering and concentrating. Any arbitrary extraction solvent can be used at that time provided it is routinely used to extract herbs.

Examples of such solvents include water, alcohols such as methanol, ethanol, propylene glycol, 1,3-butylene glycol or glycerin, water-containing alcohols and organic solvents such as chloroform, dichloroethane, carbon tetrachloride, acetone, ethyl acetate or hexane, and these can be used alone or in combination.

An extract obtained by extracting with the aforementioned solvents is used as is, after removing impurities from the concentrated extract using an adsorption method such as an ion exchange resin, or after concentrating by eluting with methanol or ethanol after adsorbing with a porous polymer column (such as Amberlite XAD-2). In addition, an extract extracted with water/ethyl acetate using a partition method can also be used.

The anti-graying agent of the present invention is applied in the form of, for example, an aqueous solution, oily liquid, other type of solution, milky liquid, cream, gel, suspension, microcapsules, powder, granules, capsules or solid preparation. After having prepared the anti-graying agent of the present invention in these formed using known methods of the prior art, the anti-graying agent can be coated, adhered, sprayed, injected or inserted into the body in the form of, for example, a lotion preparation, milky liquid preparation, cream preparation, ointment preparation, plaster preparation, poultice preparation, aerosol preparation, water-oil bilayer preparation, water-oil-powder trilayer preparation or injection preparation. There are no particular limitations on the aforementioned extract in the anti-graying agent, and the amount thereof in terms of dry weight based on the total weight of the anti-graying agent can be, e.g., 0.000001 to 5% by weight, e.g., 0.00001 to 3% by weight or 0.00001 to 1% by weight.

Among these drug forms, externally applied skin preparations such as lotion preparations, milky lotion preparations, cream preparations, ointment preparations, plaster preparations, poultice preparations and aerosol preparations are contemplated as drug forms for the object of the present invention. Furthermore, externally applied skin preparations as referred to here include prescription pharmaceuticals, over-the-counter drugs and cosmetics.

The anti-graying agent of the present invention suitably incorporates known vehicles and fragrances and the like corresponding to the desired drug form, as well as, for example, oils, surfactants, antiseptics, metal ion chelating agents, water-soluble polymers, thickeners, pigments and other powdered components, ultraviolet protectants, moisturizers, antioxidants, pH adjusters, cleansing agents, drying agents or emulsions. Moreover, other pharmaceutically active ingredients can also be incorporated in the anti-graying agent of the present invention within a range that does not impair the desired effects thereof.

Moreover, the present invention provides a method for increasing expression of AFF-4 in cells, e.g., follicular epithelial cells, using a substance selected with the aforementioned screening method, and more specifically, at least one type of herbal extract selected from the group consisting of Ganoderma lucidum (Fr.) Karst. extract, Panax schinseng Nees extract, Oryza sativa (Rice) Bran extract and Rabdosia japonicus extract. In some embodiments, the present invention provides a method for preventing graying that increases expression of AFF-4 by coating onto the scalp at least one type of herbal extract selected from the group consisting of Ganoderma lucidum (Fr.) Karst. extract, Panax schinseng Nees extract, Oryza sativa (Rice) Bran extract and Rabdosia japonicus extract in an amount effective for increasing expression of AFF-4 in cells, e.g., follicular epithelial cells. The target skin cells can be cells located at the sites of hair follicles, such as follicular epithelial cells of the scalp.

The following provides a more detailed explanation of the present invention through specific examples thereof. Furthermore, the present invention is not limited to these examples.

### EXAMPLES

### Example 1

### Method

A total of three types of vectors were prepared, i.e., an AFF-4 expression vector in which AFF-4 cDNA, having a C-Myc tag arranged in the 5′ upstream region, was linked downstream from a CMV promoter, a Foxn1 expression vector in which Foxn1 cDNA, having a Flag tag arranged in the 5′ upstream region, was linked downstream from a CMV promoter, and a luciferase vector having an Foxn1 binding region in the 5′ upstream region of an SV40 minimum promoter region and a luciferase gene in the 3′ downstream region of that promoter.

Mouse epidermal cells harvested in accordance with ordinary methods were cultured 1 to 2 days after birth. Mouse epidermal cells were prepared in which both the Foxn1 expression vector and luciferase vector were inserted into the cells (Cells 1). In addition, mouse epidermal cells were prepared in which the aforementioned AFF-4 expression vector and luciferase vector were inserted into the cells (Cells 2). Moreover, mouse epidermal cells were also prepared in which all three types of vectors (Foxn1 expression vector, AFF-4 expression vector and luciferase vector) were inserted into the mouse epidermal cells (Cells 3). Cells 1 were used in a control experiment to enable evaluation of the effect of Foxn1 on transcription activity of Foxn1 able to be evaluated using the expression level of luciferase as an indicator. Cells 2 enabled evaluation of both effects of AFF-4 on Foxn1 transcription activity. Moreover, Cells 3 enabled evaluation of effects in the presence of both Foxn1 and AFF-4 on Foxn1 transcription activity. Furthermore, the expression levels of luciferase were measured with a luminometer.

Results:

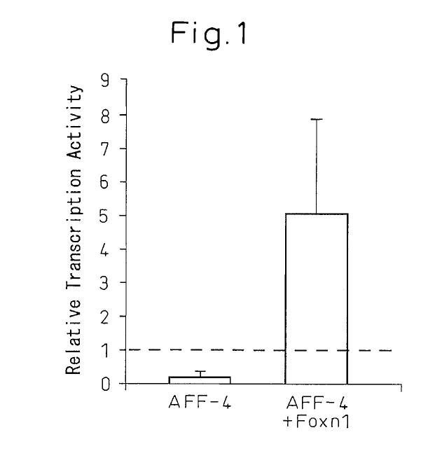

The results are shown in FIG. 1. The expression level of luciferase in the case of Foxn1 only (Cells 1) was assigned a value of “1” (indicated with a dotted line in FIG. 1). When AFF-4 was further expressed in the presence of Foxn1 (Cells 3), the expression level of luciferase increased about five-fold. However, in the case of expressing only AFF-4 (Cells 2), the expression level of luciferase decreased to about one-fifth. Accordingly, AFF-4 was confirmed to have a function that promotes transcription activity of Foxn1.

### Example 2

### Method

Mouse epidermal cells harvested from 1- to 2-day old mice were cultured followed by the preparation of mouse epidermal cells inserted with an Foxn1 expression vector in which Foxn1 cDNA, in which a Flag tag was arranged in the 5′ upstream region, was linked downstream from a CMV promoter prepared in Example 1 (Ad-Foxn1 cells), and mouse epidermal cells infected with adenovirus vector having only a Flag tag but not containing Foxn1 (Ad cells). Both cells were respectively cultured to obtain cell homogenates in accordance with ordinary methods. The cell homogenates were subjected to immunoprecipitation treatment using anti-Flag antibody, and the resulting immunoprecipitation treatment liquids were subjected to protein electrophoresis in SDS polyacrylamide gel (SDS-PAGE). Following electrophoresis, the proteins were transferred to a PVDF membrane followed by Western blotting with anti-AFF-4 antibody.

Results:

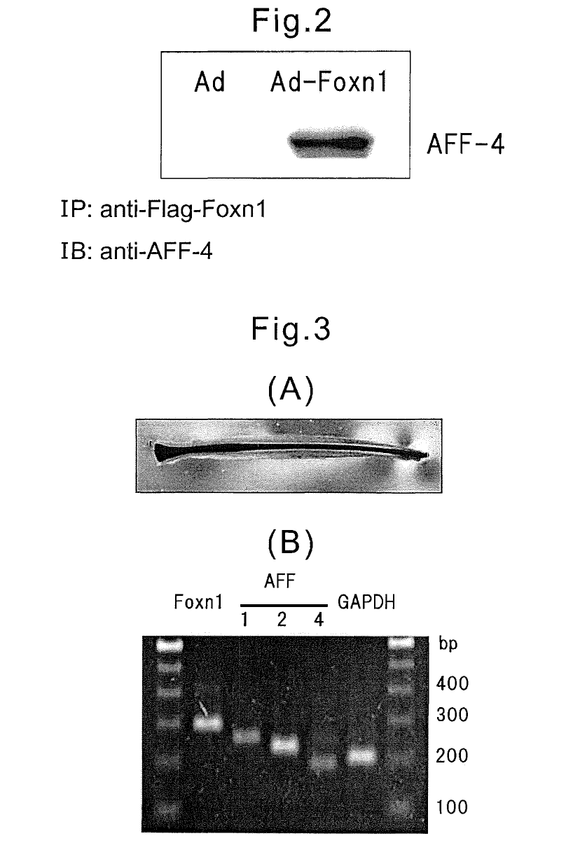

The results are shown in FIG. 2. Foxn1 having Flag protein in the cell homogenate obtained by immunoprecipitation was subjected to SDS-PAGE followed by Western blotting with anti-AFF-4 antibody. As a result, a signal was only obtained from the Ad-Foxn1 cells. This means that AFF-4 immunoprecipitated while bound to Foxn1. These results indicated that AFF-4 binds with Foxn1 within cells.

### Example 3

### Method

Anagen hair follicles as shown in FIG. 3(A) were obtained by extracting human hair. The anagen hair follicles obtained by extraction consisted of the portion comprised of follicular epithelium containing hardly any hair papilla cells. Thirty extracted hair follicles were placed in Trizol® nucleic acid preparation solution (Invitrogen) and homogenized with a homogenizer while cooling with ice to prepare total RNA in accordance with ordinary methods. A reverse transcription reaction was carried out with SuperScript® reverse transcriptase (Invitrogen) using the prepared total RNA as a template to prepare cDNA followed by carrying out PCR in accordance with ordinary methods. PCR was carried out using Taq polymerase (Takara) and the primers shown in Table 1 below. The nucleotide sequences of the primers used for PCR are shown in Table 1. PCR was carried out for a total of 30 cycles, with a single cycle consisting of 30 seconds at 94° C., 30 seconds at 60° C. and 1 minute at 72° C. Glyceraldehyde 3-phosphate dehydrogenase (GAPDH) was used for the positive control.

TABLE 1

| | | |
|---|---|---|
| Foxn1 | | |
| Forward primer: | 5′-CCT GGG TTC AGA GGT CAA AG-3′ | (SEQ ID NO: 1) |
| Reverse primer: | 5′-GGG AAG GCT CCC AGT TTT AC-3′ | (SEQ ID NO: 2) |
| AFF-1 | | |
| Forward primer: | 5′-TCC ACA GTC CCT TCC AGA AC-3′ | (SEQ ID NO: 3) |
| Reverse primer: | 5′-CGC TGT CAC TTG AAC TGC TC-3′ | (SEQ ID NO: 4) |
| AFF-2 | | |
| Forward primer: | 5′-GCC CCT AGG AAA GAA CCA AG-3′ | (SEQ ID NO: 5) |
| Reverse primer: | 5′-TGT TGC TGC CAC TGC TAC TC-3′ | (SEQ ID NO: 6) |
| AFF-4 | | |
| Forward primer: | 5′-AGA CAG TGA TGG GGA ACA GG-3′ | (SEQ ID NO: 7) |
| Reverse primer: | 5′-TGC CTC ACT GTC ACT GGA AC-3′ | (SEQ ID NO: 8) |
| bFGF | | |
| Forward primer: | 5′-CCT CAC ATC AAG CTA CAA CT-3′ | (SEQ ID NO: 9) |
| Reverse primer: | 5′-ACA CTC ATC CGT AAC ACA TT-3′ | (SEQ ID NO: 10) |
| GAPDH | | |
| Forward primer: | 5′-GAG TCA ACG GAT TTG GTC GT-3′ | (SEQ ID NO: 11) |
| Reverse primer: | 5′-TGG GAT TTC CAT TGA TGA CA-3′ | (SEQ ID NO: 12) |

Results:

The results of electrophoresis of the RT-PCR products are shown in FIG. 3(B). According to these results, Foxn1, AFF-1, AFF-2 and AFF-4 were confirmed to be expressed in human anagen hair follicles.

### Example 4

### Method

Fatty tissue was separated under a hair stereoscopic microscope from discarded scalps obtained from surgical procedures following informed consent, and hair follicles were then extracted therefrom. The extracted hair follicles were promptly embedded using OCT Compound (Tissue-Tek) followed by freezing fixation. Following fixation, the embedded hair follicles were sectioned to a thickness of 10 μm to prepare samples for staining. In the case of staining for Foxn1, rabbit IgG anti-human Foxn1 polyclonal antibody H-270 (Santa Cruz) was used for the primary antibody after diluting 50-fold. Anti-rabbit IgG-FITC was used for the color-developing secondary antibody. Furthermore, nuclei were stained with propidium iodide. Control experiments were also carried out by staining using normal rabbit IgG and without using primary antibody.

In situ hybridization was carried out according to the method described below. Probes for in situ hybridization of human Foxn1 and AFF-4 were prepared according to the following method. PCR was carried out by combining primers, in which a T7 promoter sequence was added to the 5′ upstream region of the Foxn1 and AFF-4 primers shown in Table 1, with primers to which a T7 promoter sequence was not added, so that the T7 promoter sequences were located on the 5′ ends only, to prepare DNA chains serving as templates for preparing probes for in situ hybridization.

Moreover, single-strand RNA sense and antisense chains labeled with digoxigenin were then prepared using the template DNA chains, T7 RNA polymerase and a digoxigenin labeling kit (Roche). The Foxn1 chains (GenBank: NM_{—}003593) consisted of 5′-CCTGGGTTCAGAGGTCAAAG-3′ (sense chain; SEQ ID NO:1) and 5′-GGGAAGGCTCCCAGTTTTAC-3′ (antisense chain; SEQ ID NO:2). The AFF-4 chains (GenBank: NM_{—}014423) consisted of 5′-AGACAGTGATGGGGAACAGG-3′ (sense chain; SEQ ID NO:7) and 5′-TGCCTCACTGTCACTGGAAC-3′ (antisense chain; SEQ ID NO:8). The T7 polymerase sequence was TAATACGACTCACTATAGGGAG (SEQ ID NO:13). In this experiment, blocks in which scalp samples fixed with 10% formalin were embedded in paraffin were used as test samples. The conditions of this hybridization were in accordance with the standard conditions of the in situ hybridization system (Ventana). Signal detection was carried out under color development conditions of 37° C. for 3 hours using an NBT/BCIP substrate kit (Ventana).

Results:

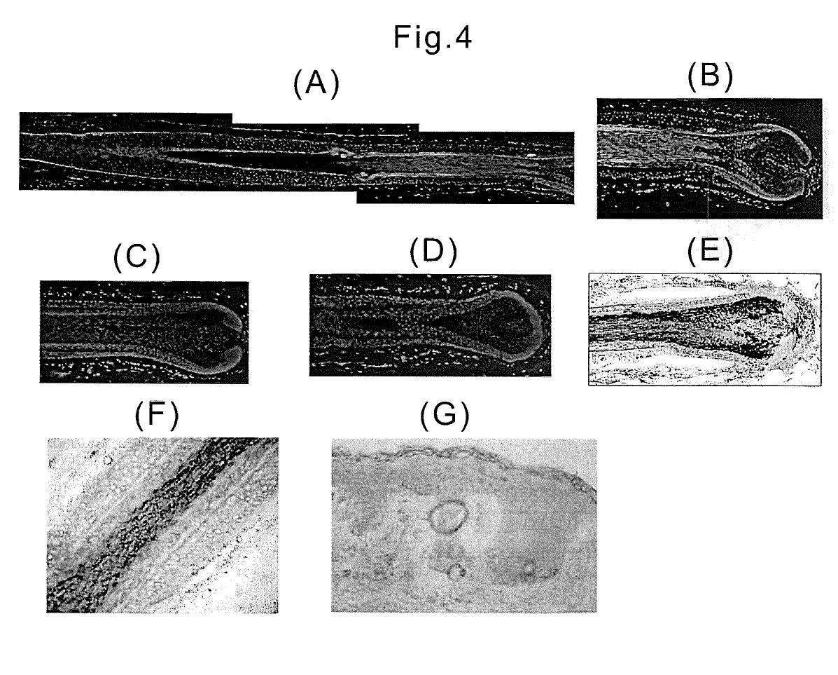

The results of staining extracted hair follicles with anti-Foxn1 antibody are shown in FIGS. 4(A) and 4(B). FIG. 4(A) depicts staining of the portion from the middle to the upper portion of the hair follicles, while FIG. 4(B) depicts staining of the lower portion thereof. According to FIGS. 4(A) and 4(B), Foxn1 was thought to be expressed in the matrix, shaft and inner root sheath in the vicinity of hair bulbs in human anagen hair follicles. FIG. 4(C) depicts staining in the case of using normal rabbit IgG instead of primary antibody, while FIG. 4(D) depicts staining in the absence of primary antibody. According to the results of FIGS. 4(C) and 4(D), the portions stained in FIGS. 4(A) and 4(B) were thought to be specially stained for Foxn1. FIG. 4(E) depicts color development following in situ hybridization with the antisense probe of Foxn1. The results were the same as those depicted in FIG. 4(B), thereby confirming by in situ hybridization as well that Foxn1 is expressed at the sites indicated above. FIGS. 4(F) and 4(G) depict color development following in situ hybridization with the antisense probe of AFF-4. The results of staining in hair follicles are shown in FIG. 4(F) while the results of staining in the skin are shown in FIG. 4(G). In contrast to the inner root sheath and shaft of the hair follicles being stained as shown in FIG. 4(F), staining of the epidermis was not observed as shown in FIG. 4(G). Based on the results shown in FIGS. 4(F) and 4(G), AFF-4 was thought to be a molecule that is expressed only in hair follicles.

### Example 5

### Method

Total RNA was prepared from thirty extracted black hair follicles under the conditions of Example 3 followed by carrying out quantitative PCR. Quantitative PCR was carried out based on a comparison of expression levels by real-time PCR using the fluorescent pigment SYBR Green I, which binds to a minor groove of double-stranded DNA with LightCycler® (Roche Diagnostics) using prepared cDNA as a template. More specifically, a reaction solution having a total volume of 20 μl (2 mM MgCl₂ and each of the forward and reverse primers indicated below at 0.25 μM each) was prepared using the LightCycler®-FastStart DNA Master SYBR Green I Kit (Roche Diagnostics) in accordance with the manual provided, and a PCR reaction was carried out with the LightCycler® (enzyme activation: 10 minutes at 95° C., thermal denaturation: 15 seconds at 95° C., annealing: 5 seconds at 58° C., elongation reaction: 10 seconds at 72° C., 40 cycles from thermal denaturation to elongation reaction) after which fluorescence intensity at completion of the elongation reaction of each cycle. This fluorescence intensity reflects the amount of PCR product at that point in time. Gene expression levels were determined based on the assumption that a PCR product Y of an initial template amount A is amplified while satisfying the relationship of Y=A×2X with respect to the number of cycles of PCR X during the exponential amplification stage of the PCR products by calculating the relative value from the number of cycles until a fixed amount of PCR product is obtained, and correcting with the expression level of glyceraldehyde 3-phosphate dehydrogenase (GAPDH). PCR was carried out using an rTaq polymerase (Takara) and the primers. The nucleotide sequences of the primers used in PCR are shown in Table 1. GAPDH was used for the positive control during PCR. PCR was carried out for a total of 30 cycles, with a single cycle consisting of 30 seconds at 94° C., 30 seconds at 60° C. and 1 minute at 72° C. Comparisons of the gene expression levels of Foxn1, AFF-1, AFF-2 and AFF-4 were respectively made in the form of relative comparisons based on the expression level of GAPDH.

Results:

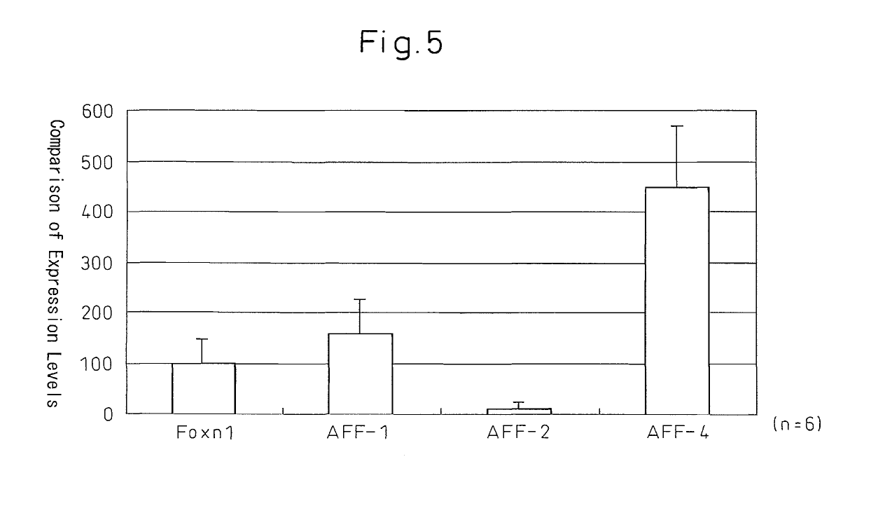

The results are shown in FIG. 5. The relative expression levels of AFF-1, AFF-2 and AFF-4 are shown in the case of assigning a value of 1.0 to the expression level of Foxn1 with respect to GAPDH. Foxn1, AFF-1 and AFF-4 were expressed in hair follicles in the black hair anagen, and the expression level of AFF-4 was particularly high. On the other hand, the expression level of AFF-2 was quite low.

### Example 6

### Method

Hairs were obtained by plucking 30 black and gray hairs each from the same volunteers. Total RNA was respectively prepared from the black hairs and gray hairs under the same conditions as Example 3 followed by carrying out quantitative PCR. Quantitative PCR was carried out in the same manner as Example 3 based on a comparison of expression levels by real-time PCR using the fluorescent pigment SYBR Green I, which binds to a minor groove of double-stranded DNA with LightCycler® (Roche Diagnostics) using prepared cDNA as a template. The DNA primers used consisted of the Foxn1, AFF-4 and bFGF primers shown in Table 1. Comparisons of gene expression levels between the black hair and gray hair were made by comparing the expression level of GAPDH of each hair with the expression level of each molecule followed by making a relative comparison between the black and gray hair. This comparison was made for each panelist and was ultimately expressed as the average of all six volunteers.

Results:

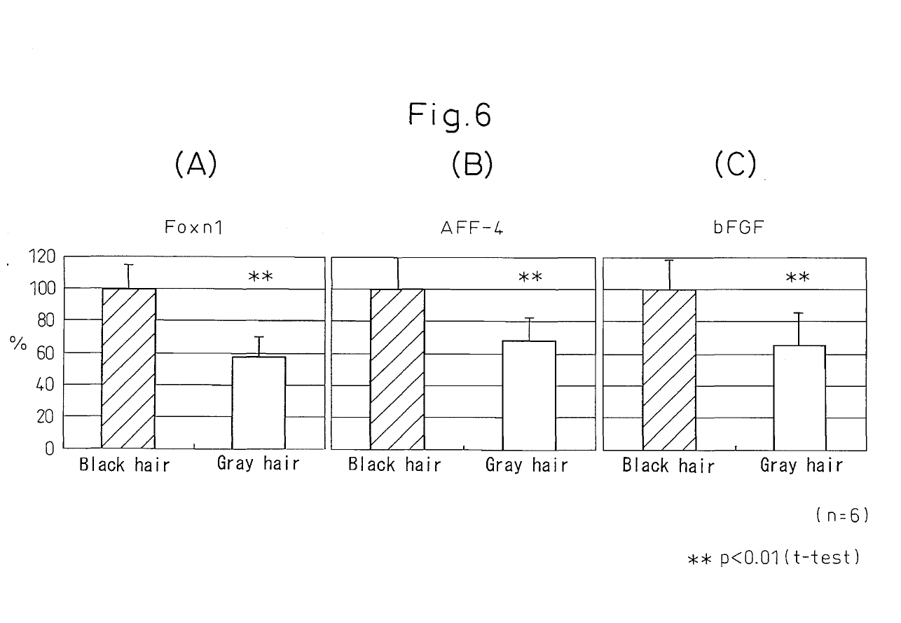

FIG. 6(A) shows a relative comparison of expression levels of Foxn1 between black and gray hair based on a value of 100 for the expression level in black hair. Similarly, FIG. 6(B) shows a relative comparison of expression levels of AFF-4 between black and gray hair, while FIG. 6(C) shows a relative comparison of expression levels of bFGF between black and gray hair. According to these results, expression of Foxn1, AFF-4 and bFGF were all lower in gray hair than in black hair. Accordingly, these three molecules were thought to have a correlation with gray hair.

### Example 7

### Method

Human follicular epithelial cells (hORS) used in this experiment were harvested and cultured in the manner described below. Fatty tissue was separated from human scalp obtained as a by-product of surgical procedures after acquiring informed consent followed by extracting hair follicles there from. After severing and removing the hair bulbs in the lower portion of the follicles using the tip of a syringe needle, the upper portions of the hair follicles were treated for 30 minutes at 37° C. with Dulbecco's Modified Eagle's Medium (DMEM, Gibco) containing 1000 U/ml of dispase (Sanko Junyaku) and 0.2% collagenase. Subsequently, the dermal sheath was removed using the tip of a syringe needle and the hair follicles were transferred to a fresh culture dish followed by treating for 5 minutes at 37° C. with phosphate buffered saline (PBS) containing 0.05% trypsin and 0.02% EDTA. Next, the hair follicles were cultured by allowing to stand undisturbed in a type 1 collagen-coated culture dish. Keratinocyte serum-free medium (Keratinocyte SFM medium, K-SFM, Invitrogen) containing all additives was used for the culturing at this time. K-SFM to which only antibiotic was added was designated as K-SFMB. The medium was replaced 4 to 5 days after the start of culturing after confirming that the hair follicles had adhered to the culture dish and that the cells had proliferated, after which the medium was replaced every two days. Cells that proliferated in this manner were treated for 5 minutes at 37° C. with 0.05 wt % trypsin and 0.02% EDTA, the reaction was stopped with an equal volume of 0.1% trypsin inhibitor, and the cells were recovered by centrifugation (800 G, 5 minutes). Next, the cells were dispersed in the aforementioned K-SFM medium and disseminated in a type 1 collagen-coated culture dish at a density of 5000 cells/cm² followed by replacing the medium every two days until the cells became confluent to obtain human outer root sheath (hORS) cells. The basic culturing conditions of these cells were in accordance with reports by Itami et al. (Itami et al. (1991) Ann. N.Y. Acad. Sci., 642: 385-395; Itami et al. (1995) Br. J. Dermatol., 132: 527-532).

The resulting hORS cells were seeded onto a type 1 collagen-coated, 24-well multiplate at 20,000 cells per well and cultured for 3 days in K-SFM followed by culturing in drug-containing K-SFMB. This culturing was carried out up to 4 hours, culturing was terminated at 1, 2, 3 and 4 hours, and mRNA was prepared with MagNA Pure LC (Roche Diagnostics). A reverse transcription reaction was carried out with SuperScript (Invitrogen) using the prepared mRNA as a template to prepare cDNA. rTaq polymerase (Takara) and the AFF-4 primers shown in Table 1 were then used to carry out PCR based on a comparison of expression levels by real-time PCR using the fluorescent pigment SYBR Green I, which binds to a minor groove of double-stranded DNA with LightCycler® (Roche Diagnostics) using the prepared cDNA as a template. Expression levels were calculated as relative values based on the expression level of glyceraldehyde 3-phosphate dehydrogenase (GAPDH).

Results:

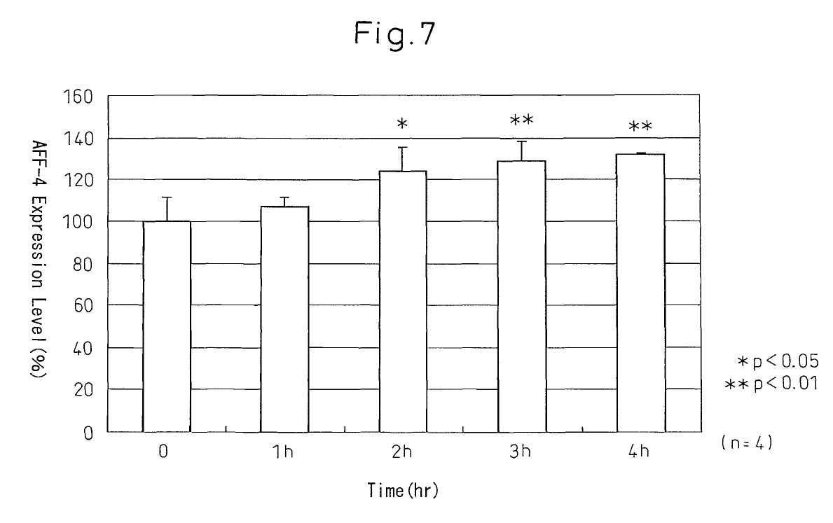

The results are shown in FIG. 7. As a result of examining the effect of Ganoderma lucidum (Fr.) Karst. extract (0.5 ppm as extract drying residue) on AFF-4 expression by hORS, Ganoderma lucidum (Fr.) Karst. extract significantly increased expression for 1 to 2 hours after addition thereof, and expression continued to increase up to 4 hours thereafter. On the basis of this finding, Ganoderma lucidum (Fr.) Karst. extract was confirmed to have a positive effect on expression of AFF-4 in hORS cells.

### Example 8

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing for 1 to 4 hours in drug-containing K-SFMB. Following culturing, mRNA and cDNA were prepared in the same manner as Example 7. rTaq polymerase (Takara) and the AFF-4 primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

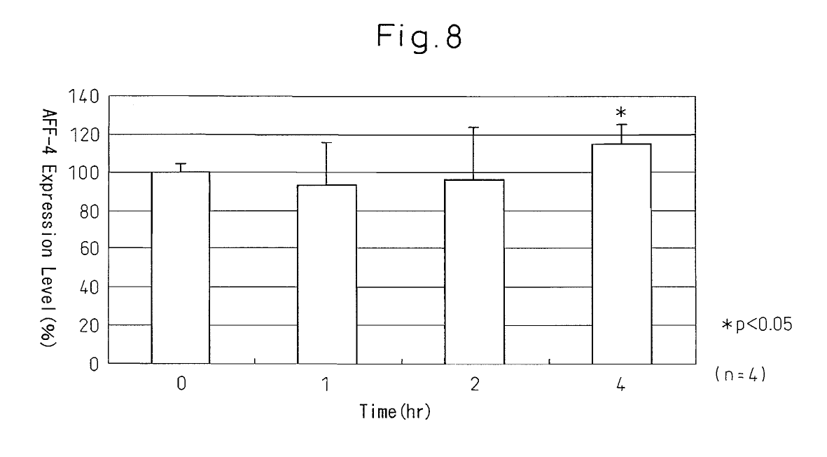

The results are shown in FIG. 8. As a result of examining the effect of Zanthoxylum piperitum extract (1.0 ppm as extract drying residue) on AFF-4 expression by hORS, Zanthoxylum piperitum extract significantly increased expression for 4 hours after addition thereof. On the basis of this finding, Zanthoxylum piperitum extract was confirmed to have a positive effect on expression of AFF-4 in hORS cells.

### Example 9

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing for 2 hours in drug-containing K-SFMB. Following culturing, mRNA and cDNA were prepared in the same manner as Example 7. rTaq polymerase (Takara) and the AFF-4 primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

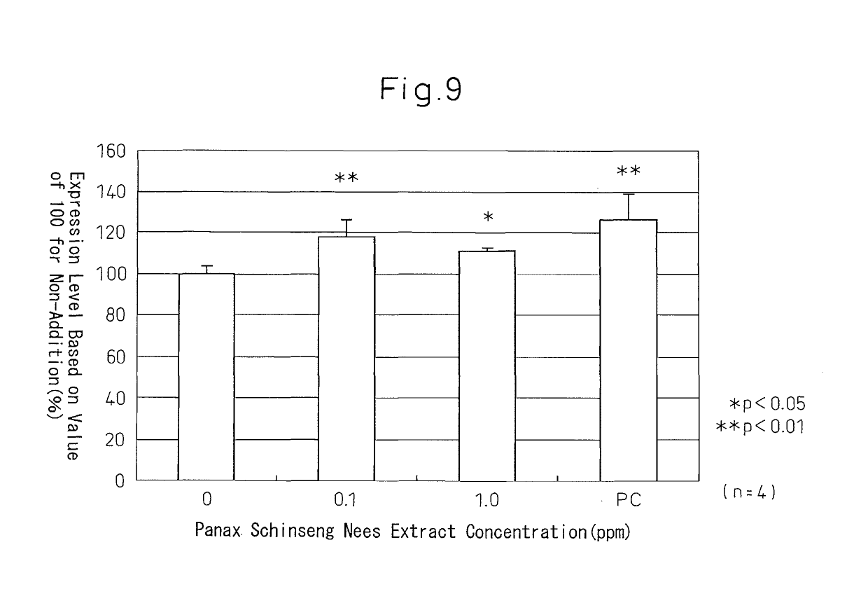

The results are shown in FIG. 9. As a result of examining the effect of Panax schinseng Nees extract (0.1 ppm and 1.0 ppm as extract drying residue) on AFF-4 expression by hORS, Panax schinseng Nees extract significantly increased expression for 2 hours after addition thereof at both 0.1 ppm and 1.0 ppm. On the basis of this finding, Panax schinseng Nees extract was confirmed to have a positive effect on expression of AFF-4 in hORS cells.

### Example 10

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing for 2 hours in drug-containing K-SFMB. Following culturing, mRNA and cDNA were prepared in the same manner as Example 7. rTaq polymerase (Takara) and the AFF-4 primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

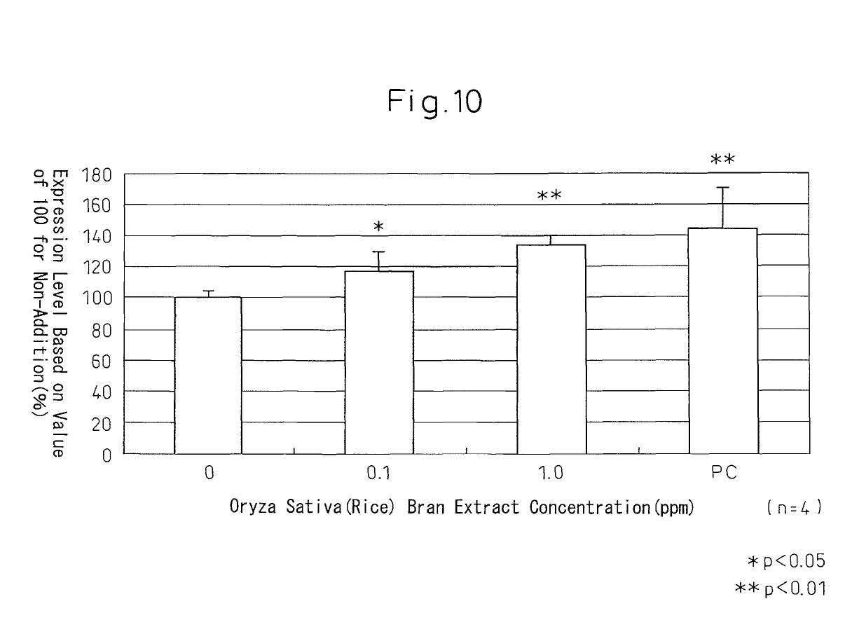

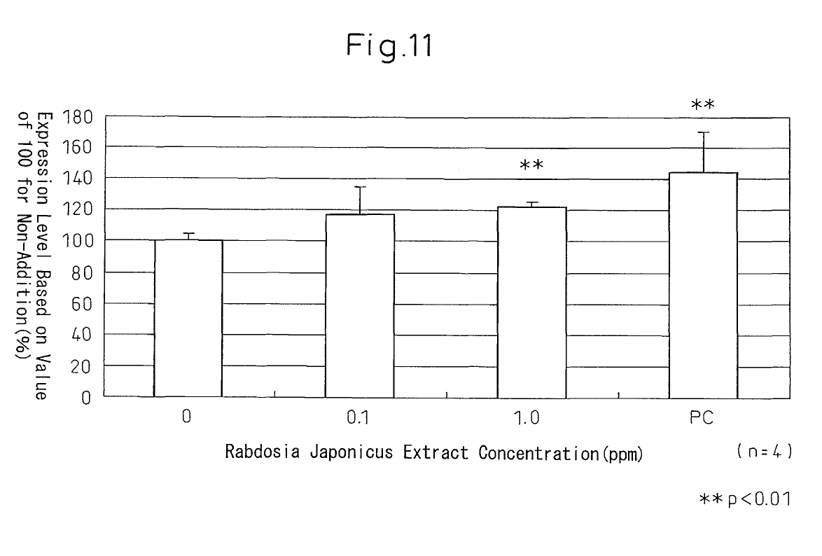

The results are shown in FIG. 10. As a result of examining the effect of Oryza sativa (Rice) Bran extract (0.1 ppm and 1.0 ppm as extract drying residue) on AFF-4 expression by hORS, Oryza sativa (Rice) Bran extract significantly increased expression for 2 hours after addition thereof at both 0.1 ppm and 1.0 ppm. On the basis of this finding, Oryza sativa (Rice) Bran extract was confirmed to have a positive effect on expression of AFF-4 in hORS cells.

### Example 11

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing for 2 hours in drug-containing K-SFMB. Following culturing, mRNA and cDNA were prepared in the same manner as Example 7. rTaq polymerase (Takara) and the AFF-4 primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

The results are shown in FIG. 11. As a result of examining the effect of Rabdosia japonicus extract (0.1 ppm and 1.0 ppm as extract drying residue) on AFF-4 expression by hORS, Rabdosia japonicus extract significantly increased expression for 2 hours after addition thereof at 1.0 ppm. On the basis of this finding, Rabdosia japonicus extract was confirmed to have a positive effect on expression of AFF-4 in hORS cells.

### Example 12

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing up to 4 hours in drug-containing K-SFMB. Following culturing, mRNA and cDNA were prepared in the same manner as Example 7. rTaq polymerase (Takara) and the bFGF primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

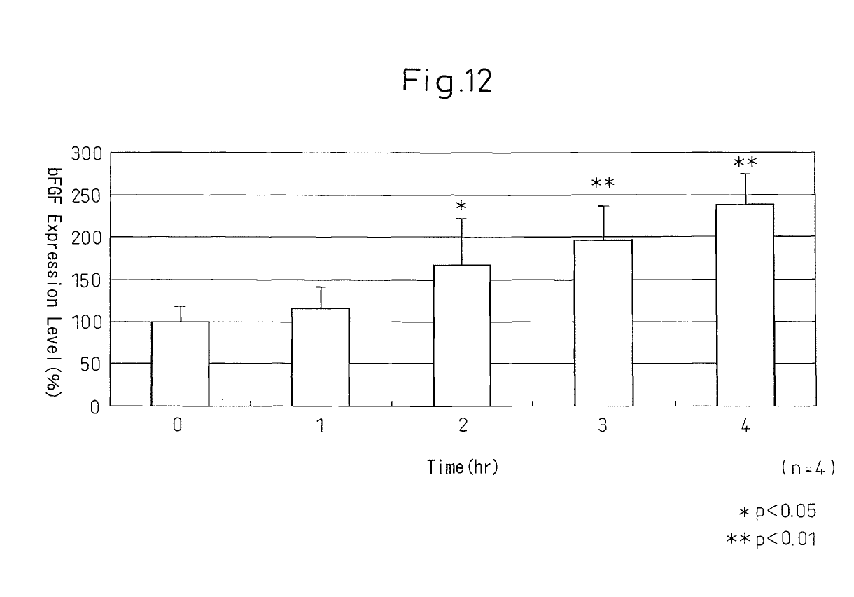

The results are shown in FIG. 12. As a result of examining the effect of Ganoderma lucidum (Fr.) Karst. extract (1.0 ppm as extract drying residue) on bFGF expression by hORS, Ganoderma lucidum (Fr.) Karst. extract significantly increased expression for 3 hours or more after addition thereof. On the basis of this finding, Ganoderma lucidum (Fr.) Karst. extract was confirmed to have a positive effect on expression of bFGF in hORS cells.

### Example 13

### Method

hORS cells were harvested and cultured using the same method as that described in Example 7. The resulting hORS cells were seeded onto a type 1 collagen-coated, 24-well multiplate at 20,000 cells per well and cultured for 3 days in K-SFM followed by culturing up to 4 hours in drug-containing K-SFMB. Following culturing, mRNA and cDNA were prepared in the same manner as Example 7. rTaq polymerase (Takara) and the bFGF primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH). Cells cultured in K-SFMB medium not containing extract were used as a negative control, while cells cultured in K-SFM containing all factors were used as a positive control. Each experiment was conducted four times each.

Results:

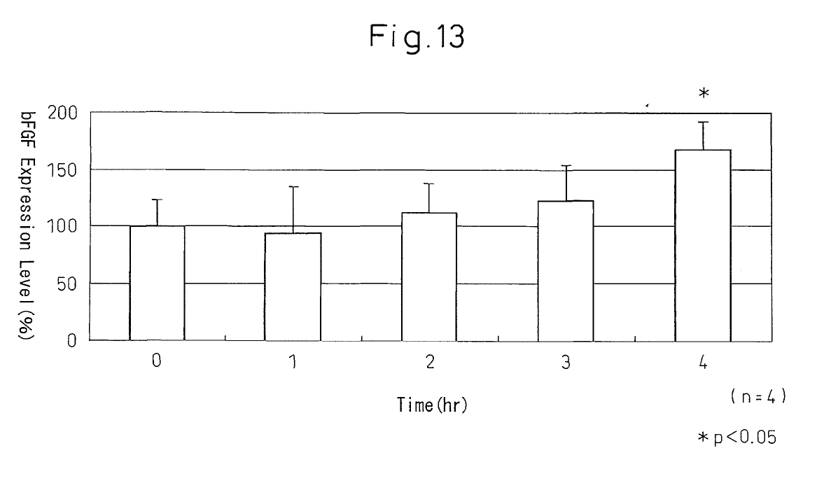

The results are shown in FIG. 13. As a result of examining the effect of Zanthoxylum piperitum extract (1.0 ppm as extract drying residue) on bFGF expression by hORS, Zanthoxylum piperitum extract significantly increased expression for 4 hours after addition thereof. On the basis of this finding, Zanthoxylum piperitum extract was confirmed to have a positive effect on expression of bFGF in hORS cells.

### Example 14

### Method

Human epidermal cells (HaCaT) were seeded onto a type 1 collagen-coated, 24-well multiplate at 20,000 cells per well and cultured for 4 days in K-SFM followed by culturing for up to 3 hours in drug-containing K-SFMB. Following culturing, mRNA and cDNA were prepared in the same manner as Example 7. rTaq polymerase (Takara) and the bFGF primers shown in Table 1 were then used to carry out PCR based on a comparison of expression levels by real-time PCR using SYBR Green I with LightCycler® (Roche Diagnostics) using the prepared cDNA as a template. Expression levels were calculated as relative values based on the expression level of glyceraldehyde 3-phosphate dehydrogenase (GAPDH).

Results:

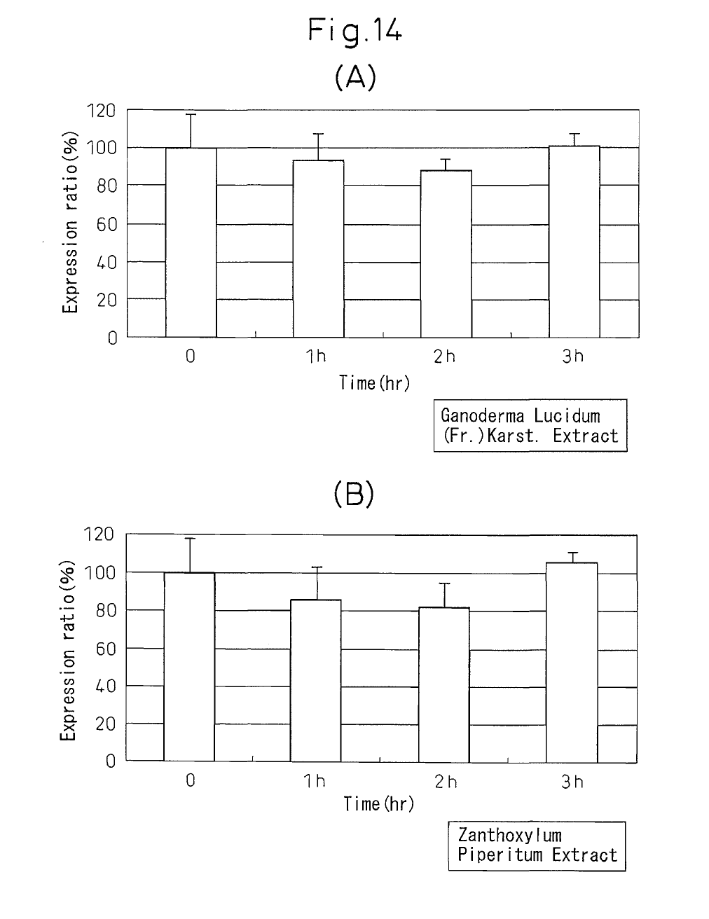

As a result of examining the effects of Ganoderma lucidum (Fr.) Karst. extract (FIG. 14(A)) and Zanthoxylum piperitum extract (FIG. 14(B)) (1.0 ppm as extract drying residue) on bFGF expression by HaCaT, significant changes in the expression levels of bFGF were not observed even at 3 hours after addition for either of the extracts. On the basis of these findings, Ganoderma lucidum (Fr.) Karst. extract was confirmed to not have an effect on expression of bFGF in human epidermal cells.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method for screening anti-graying agent, comprising: selecting substances that increase expression of AFF-4 in cells by applying a candidate substance to the cells.

2. The method according to claim 1, wherein the cells are follicular epithelial cells.

3. The method according to claim 1, wherein the amount of mRNA encoding AFF-4 extracted from the cells is measured by PCR.

4. The method according to claim 2, wherein the amount of mRNA encoding AFF-4 extracted from the cells is measured by PCR.

5. An anti-graying agent containing at least one type of herbal extract selected from the group consisting of Ganoderma lucidum (Fr.) Karst. extract, Panax schinseng Nees extract, Oryza sativa (Rice) Bran extract and Rabdosia japonicus extract in an amount effective for increasing expression of AFF-4.

6. A method for increasing expression of AFF-4 using at least one type of herbal extract selected from the group consisting of Ganoderma lucidum (Fr.) Karst. extract, Panax schinseng Nees extract, Oryza sativa (Rice) Bran extract and Rabdosia japonicus extract.

7. A method for preventing or inhibiting gray hair comprising: increasing expression of AFF-4 by coating onto the scalp at least one type of herbal extract selected from the group consisting of Ganoderma lucidum (Fr.) Karst. extract, Panax schinseng Nees extract, Oryza sativa (Rice) Bran extract and Rabdosia japonicus extract in an amount effective for increasing expression of AFF-4.

8. A method of screening for an anti-graying agent, the method comprising:
contacting a cell with a test substance;
measuring expression of AFF-4 in the cell; and
selecting the test substance as a candidate anti-graying agent if it increases expression of AFF-4 in the cell as compared to expression of AFF-4 in the absence of the test substance.

9. The method according to claim 8, wherein the cell is a follicular epithelial cell.

10. The method according to claim 1, wherein measuring expression of AFF-4 in the cell comprises measuring AFF-4 mRNA expression by PCR.

11. The method according to claim 2, wherein measuring expression of AFF-4 in the cell comprises measuring AFF-4 mRNA expression by PCR.

12. A method for increasing expression of AFF-4 comprising administering at least one type of herbal extract selected from the group consisting of Ganoderma lucidum (Fr.) Karst. extract, Panax schinseng Nees extract, Oryza sativa (Rice) Bran extract and Rabdosia japonicus extract.

13. A method for preventing or inhibiting hair graying comprising administering at least one type of herbal extract selected from the group consisting of Ganoderma lucidum (Fr.) Karst. extract, Panax schinseng Nees extract, Oryza sativa (Rice) Bran extract and Rabdosia japonicus extract in an amount effective for increase expression of AFF-4.
